(19) United States
(12) Patent Application Publication
(10) Pub. No.: US 2023/0402138 A1
(43) Pub. Date: Dec. 14, 2023

(54) Electronic Goal Attainment

(71) Applicants: Kenneth Rockwood, Halifax (CA); Aaqib Shehzad, Halifax (CA); Amanda England, Halifax (CA); Chere Chapman, Halifax (CA)

(72) Inventors: Kenneth Rockwood, Halifax (CA); Aaqib Shehzad, Halifax (CA); Amanda England, Halifax (CA); Chere Chapman, Halifax (CA)

(21) Appl. No.: 17/838,208

(22) Filed: Jun. 11, 2022

(51) Int. Cl. G16H 10/20; G16H 40/20; G16H 20/10

(52) U.S. Cl. CPC G16H 10/20; G16H 40/20; G16H 20/10; G16H 10/60

(57) ABSTRACT

A patient care management system and method coupled to the Internet for using goal attainment scaling diagnosis methodologies for the treatment of diseases in test subjects is described. The patient care management system may include an Internet-based multi-tenanted electronic data collection platform for hosting and coordinating multiple clinical trials of test subjects. The platform may be used independently or in collaboration with third-party data collection tools. The platform may include a number of interconnected modules that allow the users to configure any number of clinical trials.

### TECHNICAL FIELD

The present disclosure relates broadly to patient care management and clinical drug development. More particularly, the present disclosure relates to using goal attainment scaling diagnosis methodologies to treat diseases and test effectiveness of new therapeutic interventions in test subjects.

### BACKGROUND

Goal Attainment Scaling (GAS) methodology is an individualized patient-reported outcome that quantifies the effects of an intervention based on personal goals. Since its introduction in the 1960s, GAS methodology has been implemented in clinical trials in numerous areas of study, and has proven to be a valid, reliable, and sensitive instrument for measuring personally meaningful change. It is particularly well-suited to evaluating interventions for conditions with high heterogeneity in symptoms and progression, where standardized outcomes often fall short. GAS methodology can be applied to any number of conditions to be tracked as a function of time, such as tracking health conditions, including tracing dementia, ADHD, elderly care settings, chronic pain, cognitive rehabilitation, amputee rehabilitation, and child development motor skills.

Despite its proven benefits, GAS has generally been underutilized in clinical trials and clinical care delivery. The primary reason is that it can be difficult and time-consuming to identify appropriate treatment goals and construct full scales with descriptive attainment levels. This can especially be true when applying GAS to a new disease area or patient population, with little qualitative research into potential goal areas. Inappropriate goals and poorly constructed attainment scales can lead to negative study results or biased interpretations. In addition, the current crop of electronic data capture platforms used in clinical trials lack sufficient functionality and support for capturing GAS-based outcomes. This is due mainly to the unique nature of GAS that requires anchor-based longitudinal capture of data.

### SUMMARY

Patient care management systems and techniques coupled to the Internet for using GAS diagnosis methodologies for the treatment of diseases and test effectiveness of new therapeutic inteerventions in test subjects are described. The patient care management system may include an Internet-based multi-tenanted electronic data collection platform for hosting and coordinating multiple clinical trials or clinical care delivery centers of test subjects. The platform may be used independently or in collaboration with third-party data collection tools. The platform may include a number of interconnected modules that allow the users to configure adaptable graphical interfaces for n number of clinical trials, clinical delivery centers or patient interaction portals.

In its most basic configuration, the presently disclosed innovation may be used as an electronic data collection and clinical care management tool for GAS in multicenter clinical trial research, hospitals, and as a tool to capture patient-reported outcome measures.

As compared to traditional paper-based goal attainment scaling, the presently disclosed software schemes and methods are configured to allow system configurators to program context dependent series of questions and workflows that guide users by way of an electronic user interface that helps the users to easily identify, describe, rank, and track clinical test subjects' attainment of their treatment goals. This information is captured and stored electronically and may be shared worldwide via networking on the Internet.

The presently disclosed innovation provides and discloses Internet networking software schemes and methods that can be programmed to offer the users a quick and easy menu of treatment goals/symptoms to select from relevant to the disease area or domain.

Also, the presently disclosed innovation provides and discloses Internet networking software schemes and methods that may be customized and used in different domains, including but not limited to clinical trials, hospitals, and clinic facilities.

### BRIEF DESCRIPTION OF THE DRAWINGS

In order to clearly illustrate the technical solution of the embodiments of the disclosure, the drawings of the embodiments will be briefly described in the following; the described drawings are only related to some embodiments of the disclosure and thus are not limitative of the disclosure.

FIG. 1 depicts a schematic arrangement of a plurality of interactive modules that are operatively coupled together within a single server containing the platform according to one embodiment.

FIG. 2 depicts a stylized arrangement of various users interacting with a network of servers within the Internet containing the platform, according to one embodiment.

### REFERENCE NUMBERS

10—patient care management (PCM) system;
11—platform;
12—test subject;
14—Internet cloud;
16—clinical trials;
18—third-party data collection tool;
20—customer identity access management (CIAM) module;
22—users;
24—system administrator (SYA) module;
26—study planning (STP) module;
28—site management (SMG) module;
30—form builder (FBR) module;
32—study protocol (SPR) module;
34—study user management (STUM) module;
36—data collection (DCN) module;
38—graphical user interface (GUI);
40—form display (FDY) submodule;
42—signature (SIGN) submodule;
44—query (QRY) submodule;
46—server;
48—database,

### DETAILED DESCRIPTION

In order to make objects, technical details, and advantages of the embodiments of the disclosure apparent, the technical solutions of the embodiments will be described in a clearly and fully understandable way in connection with the drawings related to the embodiments of the disclosure. It is understood that the described embodiments are just a part but not all of the embodiments of the disclosure. All other embodiments which may be obtained by one skilled in the art without any creative labor based on the described embodiments of the present disclosure fall within the scope of the present disclosure.

In the present disclosure, unless specified or limited otherwise, the term “coupled,” and the like are used broadly, and may be, for example, fixed connections, detachable connections, software connections, or integral connections; may be direct connections or indirect connections via intervening elements; which can be understood by those skilled in the art according to specific situations.

In its most basic configuration, one embodiment presently disclosed may be a PCM System 10, which may be configured to use a goal attainment scaling methodology for treating or diagnosing a disease in Test Subjects 12. PCM System 10 may be a computation-based electronic data capture tool for use in centralized or decentralized trials. PCM System 10 may incorporate Software Platform 11 coupled to any number of Servers 46 on Internet Cloud 14, so that Platform 11 may be used independently or in collaboration with Third-party Data Collection Tools 18. Internet Cloud 14 connection to Platform 11 may provide a multi-tenanted electronic data collection networking scheme that hosts and coordinates multiple Clinical Trials 16 of Test Subjects 12. Platform 11 may include but is not limited to CIAM Module 20, System Administrator (SYA) Module 24, Study Planning (STP) Module 26, Site Management (SMG) Module 28, Form Builder (FBR) Module 30, Study Protocol (SPR) Module 32, Study User Management Module 34, and Data Collection (DCN) Module 36.

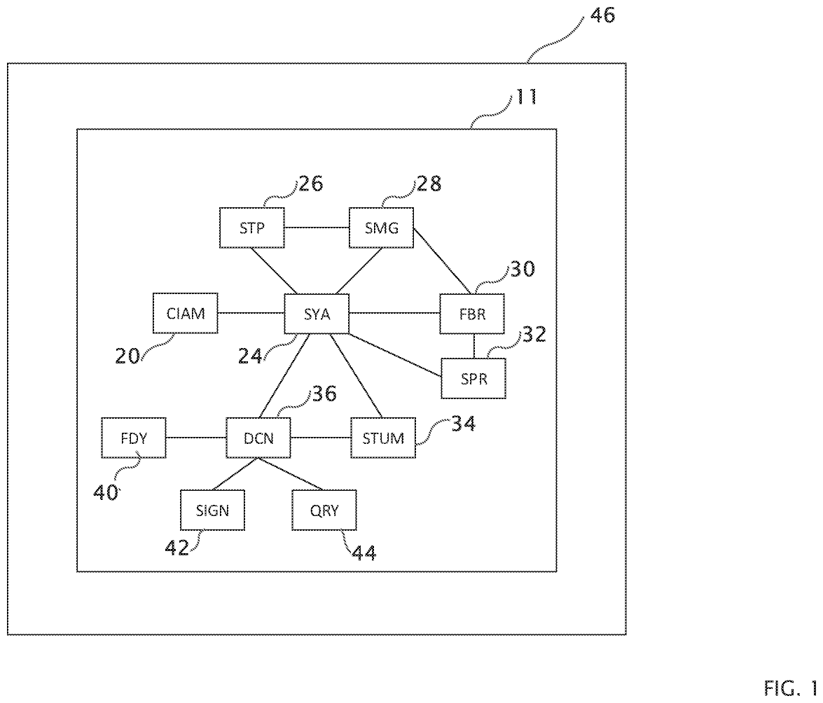

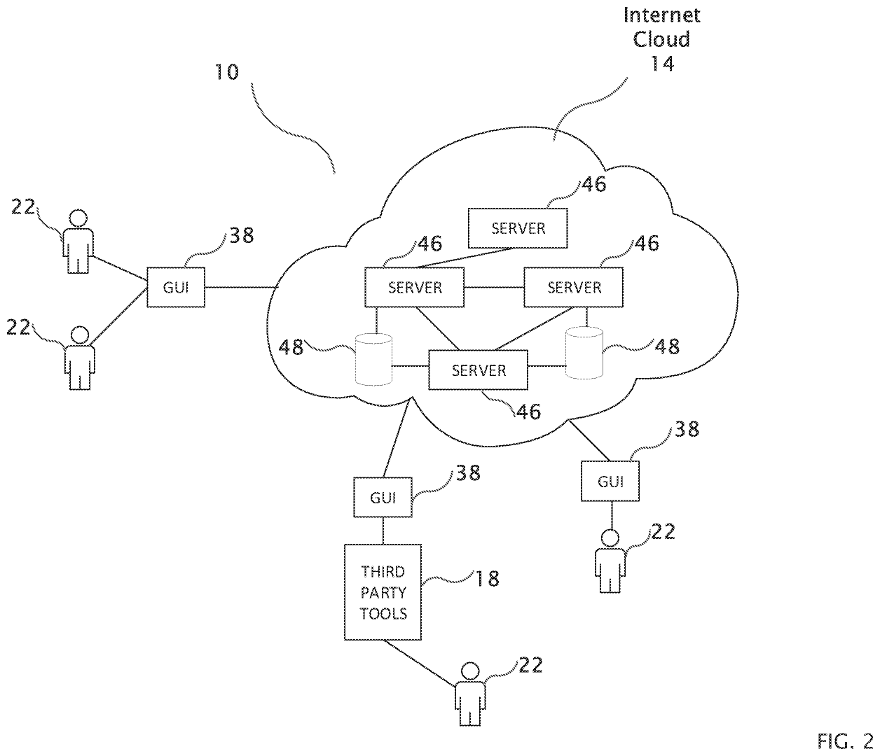

As illustrated in FIG. 1, CIAM 20, SYA 24, STP 26, SMG 28, FBR 30, SPR 32, STUM 34, and DCN 36 modules may be operatively coupled to each together within Single Server 46 containing Platform 11. Alternately as shown in FIG. 2, CIAM 20, SYA 24, STP 26, SMG 28, FBR 30, SPR 32, STUM 34, and DCN 36 modules may be operatively coupled to each together within a network of Servers 46 in Internet Cloud 14 that distributes portions of Platform 11 throughout the network of Servers 46 in Internet Cloud 14.

These CIAM 20, SYA 24, STP 26, SMG 28, FBR 30, SPR 32, STUM 34, and DCN 36 modules can be coupled to each other in any number of different ways so long as they are all operatively coupled to each together.

CIAM Module 20 of Platform 11 may be configured to authorize selective access to Users 22 onto Platform 11. Users 22 may include System Administrator Users 22, Clinical Trial Designer Users 22, Data Entry Personnel Users 22, and Data Study Reviewer Users 22.

System Administrator (SYA) Module 24 of Platform 11 may be configured to host and coordinate multiple Clinical Trials 16 by providing a workspace and access to databases 48 associated with the multiple clinical trials 16.

The study planning (STP) Module 26 of Platform 11 may be configured to manage the databases 48 in which at least one of the databases 48 includes initial details and clinical details.

Screening inquiries for potential test subjects to be included in the subsequent clinical study are preferably recorded in the database to include any number of different and probing inquiries. For example, screening inquiries or clinical details for mild to moderate dementia may include but are not limited to the queries of (1) Anxiety and worry, (2) decreased interest or initiative, (3) delusions and paranoia, (4) disorientation to time, (5) hallucinations, (6) impaired attention or concentration, (7) impaired comprehension or understanding, (8) impaired judgment, (9) language problems, (10) problems with recent memory, (11) misplacing or losing objects, (12) needs help with dressing, (13) problems with past memory, (14) problems with financial management, (15) problems with household chores, (16) problems with insight or awareness, (17) problems with telephone use, (18) repetitive questions and stories, (19) sleep disturbances, and (20) social interaction or withdrawal.

The initial details may include but are not limited to a title field, a brief description field, and a language field. The clinical details may include, but are not limited to, an inclusion/exclusion criteria field, a study site field, a measurement unit field, a code list field, a forms field, a study event field, a study protocol field, and a user field. Complete customization of codes and symptoms may also be supported.

Some representative examples of the inclusion criteria fields may include but are not limited to the present disclosure can be the following exemplary queries of (1) Unable to ambulate without assistance, (2) Unable to dress without assistance, (3) Unable to bathe without assistance, (4) Urinary or fecal incontinence intermittent or constant, and (5) No consistent meaningful verbal communication, speech may be limited to six or fewer intelligible words or only stereotypical phrases, and community-based observational research.

Some representative examples of the exclusion criteria fields may include but are not limited to the present disclosure can be the following queries, for example, of (1) Aspiration pneumonia, (2) Pyelonephritis or upper urinary tract infection, (3) Septicemia, (4) Decubitus ulcers, multiple, stage 3-4, (5) Fever, recurrent after antibiotics, and (6) Inability to maintain sufficient fluid and calorie intake with 10% weight loss during the previous six months or serum albumin <2.5 gm/dl.

Some examples of code list fields include but are not limited to following of (1) adverse experiences action, (2) adverse experiences drug relationship, (3) adverse experiences experience course, (4) adverse experiences intensity, (5) adverse experiences outcome, (5) ethnicity, (6) gender, and (7) yes/no code list. Another example may be requiring subjects to be a certain age to participate in a study,

Some examples of the forms field may include any number of different forms such as traditional case report forms and GAS forms. For example, the GAS forms may include (1) Mild-Moderate GAS Form where the menu may be based on GAS for Mild-Moderate Dementia, (2) Traditional GAS Form where there are open-ended GAS criteria listed, and (3) Demo GAS form. These forms may include (1) eligibility criteria, (2) demographics, and (3) adverse experiences data type record, such as a string using medical terminology observed or elicited by the following direct questions to the patient, provide the diagnosis, not symptoms where possible (4) a study event field, (5) a study protocol field in a coded value format, and a user field.

The site management (SMG) Module 28 of Platform 11 may be configured to allow selectively authorized Users 22 to test the effectiveness of the specific interventions and managing disease in Test Subjects 12.

The form builder (FBR) Module 30 of Platform 11 may be configured to allow the clinical trial designer Users 22 to design CRFs for the multiple clinical trials 16. FBR Module 30 may allow Clinical Trial Designer Users 22 to set workflow of Multiple Clinical Trials 16, identify goals of Test Subjects 12, define attainment levels of the goals, rank the goals, and follow up on rankings of the goals. Ranking and scoring criteria may include, for example, criteria of (1) a rank criteria of “much better” having a score of “+2”, (2) a rank criteria of “somewhat better” having a score of “+1”, (3) a rank criteria of “baseline” having a score of “0”, (4) a rank criteria of “somewhat worse” having a score of “−1”, and (5) a rank criteria of “much worst” having a score of “−2”. The CRFs allow longitudinal data collection of goals and symptoms of Test Subjects 12 participating in the multiple clinical trials 16 by capturing point in time data. FBR Module 30 may allow Clinical Trial Designers Users 22 to configure the workflow of a GAS form or an equivalent patient-centered outcome measure. The workflow may include identifying goals from an established menu of goals or symptoms to choose from or entering a new goal or symptom not included in the established menu, setting attainment criteria of goals, and follow-up rating of goal attainment. FBR Module 30 may also allow Clinical Trial Designer Users 22 to configure the type, frequency, and content of the programable prompts presented to Test Subject 12 or Users 22 completing a GAS specification during an initial visit (first interaction) or follow-up visits (follow-up interactions).

Study Protocol (SPR) Module 32 of Platform 11 may be configured to allow Clinical Trial Designer Users 22 to define scheduled and unscheduled study visits for Test Subjects 12 participating in the multiple clinical trials 16 and to collect data associated with Test Subjects 12 during the study visits. Study Protocol (SPR) Module 32 may also allow Clinical Trial Designers Users 22 to establish a sequence of events and rules to establish timing of those events.

The Study User Management Module 34 of Platform 11 may be configured to allow Clinical Trial Designer Users 22 to add and assign Data Entry Personnel Users 22 for reading or writing data in Databases 48 and allow access to Database 48 to Data Study Reviewer Users 22.

Data Collection (DCN) Module 36 of Platform 11 may be configured to provide GUI 38 for use by Data Entry Personnel Users 22 and Data Study Reviewer Users 22. The DCN Module 36 may be configured to allow Data Entry Personnel Users 22 to add Test Subjects 12 and to collect data from the CRFs. Accordingly, DCN Module 36 may be a primary point of interaction for Data Entry Personnel Users 22 to add Test Subjects 12 and collect or enter data in Databases 48 of Multiple Clinical Trials 16 and in the CRFs.

The DCN Module 36 of Platform 11 may also include various submodules, which may include but are not limited to form display (FDY) Submodule 40, signature (SIGN) Submodule 42, and a query (QRY) Submodule 44. As illustrated in FIG. 1, the FDY 40, SIGN 42, and QRY 44 Submodules of the DCN Module 36 are all operatively coupled. These FDY 40, SIGN 42, and QRY 44 Submodules can be coupled to each other in any number of different ways so long as they are all eventually operatively coupled to each other.

The FDY Submodule 40 of the DCN Module 36 may be configured to display data in GAS format so as to present an interactive GUI 38 that guides Users 22 through the workflow of identified goals and to guide Users 22 through programmed prompts to assess progress of Test Subjects 12 in achieving their respective identified goals in subsequent follow-up visits.

The SIGN Submodule 42 of the DCN Module 36 may be configured to allow Users 22 to attach a digital signature in the databases 48 associated with the Test Subjects 12, the CRFs, and the study visits. The SIGN Submodule 42 may be configured to require Users 22 to revalidate and authenticate Users 22. Upon successful revalidation and authentication, the SIGN Submodule 42 may present a legal disclaimer to the Users 22 prior to Users 22 signing into the databases 48. An example of the legal disclaimer may be, “I warrant the truthfulness of the electronic data for this subject are a full, accurate and complete record of the observations recorded. I acknowledge and intend for this electronic signature to be the legally binding equivalent of my written signature.” As a result, SIGN Submodule 42 may provide an on-demand guarantee of fidelity of data in the databases 48.

The QRY Submodule 44 of DCN Module 36 may be configured to allow Data Entry Personnel Users 22 to annotate data in the databases 48 that fails any quality benchmark set in the multiple clinical trials 16.

Diseases diagnosed in Test Subjects 12 by PCM System 10 may include any number of different diseases such as dementia, depression, bipolar disorder, eating disorder, post-traumatic stress disorder, psychotic disorder, autism, generalized anxiety disorder, panic disorder, phobia, social anxiety disorder, mental health disorder, obsessive-compulsive disorder, oncology, blood disorders, psychological diseases, delirium, delusional disorder, mood disorders, substance abuse, rare diseases, and medical-neurologic conditions, for example.

Some of these treatments for diseased diagnosed Test Subjects 12 by PCM System 10 can include any number of different therapeutic treatments such as prescribing medications to Test Subjects 12 or providing nursing care. Some of the medications for treating the various disease include but are not limited to buspirone, trazodone, valproic acid, carbamazepine, gynecomastia, donepezil, galantamine, rivastigmine, memantine, aripiprazole, clozapine, haloperidol, olanzapine, quetiapine, risperidone, ziprasidone, Vyvanse, Ritalin, Adderall, and Dexedrine.

Another embodiment of the disclosure may be a GAS method for diagnosis and treatment of a disease in Test Subjects 12 The presently disclosed GAS method can include the operations of hosting and coordinating multiple Clinical Trials 16 of Test Subjects 12 by using Internet Cloud 14 based multi-tenanted electronic data collection platform 11 that can be used independently or used in collaboration with Third-Party Data Collection Tools 18, diagnosing the disease, and treating the disease.

The operation of hosting and coordinating multiple clinical trials 16 of Test Subjects 12 may use Internet Cloud 14 based multi-tenanted electronic data collection Platform 11 (as described above) so that Platform 11 can be used independently or in collaboration with third-party data collection tools 18.

Platform 11 may be used for any number of diseases such as Parkinson's, Alzheimer's, Lupus, Hutchinson disease, Huntington's, infectious disease, Kawasaki disease, Lyme disease, cerebrovascular disease, Erdheim-Chester disease, cognitive impairment disease, coronavirus disease, coronary artery disease, infectious disease, Wilson's disease, celiac disease, cancer disease, mad cow disease, malignant disease, melanoma disease, meningitis disease, schizophrenia disease, anxiety disorder disease, depression. Other diseases include neurological diseases such as dementia, depression, bipolar disorder, eating disorder, post-traumatic stress disorder, psychotic disorder, autism, generalized anxiety disorder, panic disorder, phobia, social anxiety disorder, mental health disorder, obsessive-compulsive disorder, delirium, delusional disorder, mood disorders, substance abuse, and medical-neurologic conditions. Platform 11 may be used for chronic and complex disease areas, including, for example, kidney disorders, hemophilia, or oncology.

The treatment operation of the disease may be any therapeutic treatment such as prescribing medications to Test Subjects 12 or providing nursing care to Test Subjects 12. The prescribed medications include any number of different medications, for example, buspirone, trazodone, valproic acid, carbamazepine, gynecomastia, donepezil, Vyvanse, galantamine, rivastigmine, memantine, aripiprazole, clozapine, haloperidol, olanzapine, quetiapine, risperidone, ziprasidone, Ritalin, Adderall, and Dexedrine.

What is described above may be related to the illustrative embodiments of the invention only and not limitative to the scope of the invention; the scope of the invention is defined by the accompanying claims.

We claim:

1. A patient care management system using a goal attainment scaling method for treatment of a disease in test subjects, the patient care management system comprising:
an Internet-based multi-tenanted electronic data collection platform for hosting and coordinating multiple clinical trials of test subjects in which the platform can be used independently or used in collaboration with third-party data collection tools, the platform comprising:
a customer identity access management module configured to authorize selective access to users onto the platform, wherein users include: system administrator users, clinical trial designer users, data entry personnel users, and data study reviewer users;
a system administrator module configured to host and to coordinate multiple clinical trials by providing a workspace and access to databases associated with the multiple clinical trials;
a study planning module configured to manage the databases;
a site management (SMG) module configured to allow selectively authorized users to establish information about the multiple clinical trials and to link test subjects to the multiple clinical trials;
a form builder module configured to allow the clinical trial designer users to design case report forms for the multiple clinical trials;
a study protocol module configured to allow the clinical trial designer users to define scheduled and unscheduled study visits for test subjects participating in the multiple clinical trials and to collect data associated with test subjects during the study visits;
a study user management module configured to allow the clinical trial designer users to add and assign data entry personnel users to read or write data in the databases and to enable access to the database to the data study reviewer users; and
a data collection module configured to provide a graphical user interface for use by the data entry personnel users and the data study reviewer users,
wherein the customer identity access management, system administrator, study planning, SMG, form builder, study protocol, study user management, and data collection modules are operatively coupled together.

2. The patient care management system of claim 1, wherein at least one of the databases includes initial details and clinical details.

3. The patient care management system of claim 2, wherein initial details include: a title field, a brief description field, and a language field.

4. The patient care management system of claim 2, wherein clinical details include: an inclusion/exclusion criteria field, a study site field, a measurement unit field, a code list field, a forms field, a study event field, a study protocol field, and a user field.

5. The patient care management system of claim 1, wherein the form builder module allows the clinical trial designer users to set workflow of the multiple clinical trials, to identify goals of the test subjects, to define attainment levels of the goals, to rank the goals, and to follow up on rankings of the goals.

6. The patient care management system of claim 1, wherein the case report forms allow longitudinal data collection of goals and symptoms of the test subjects participating in the multiple clinical trials by capturing point in time data.

7. The patient care management system of claim 1, wherein the data collection module is configured to allow data entry personnel users to add test subjects and to collect data from the case report forms.

8. The patient care management system of claim 7, wherein the data collection module is a primary point of interaction for the data entry personnel users to add test subjects and to collect or enter data in the databases of the multiple clinical trials and in the case report forms.

9. The patient care management system of claim 1, wherein the data collection module comprises a form display submodule; a signature submodule; and a query submodule in which the form display, signature, and query submodules are operatively coupled to each other.

10. The patient care management system of claim 9, wherein the form display, signature, and query submodules of the data collection module are operatively coupled to each other.

11. The patient care management system of claim 9, wherein the form display submodule is configured to display data in GAS format so as to present an interactive GUI that guides users through a workflow of identified goals and to guide users through programmed prompts to assess progress of test subjects in achieving their respective identified goals in subsequent follow-up study visits.

12. The patient care management system of claim 10, wherein the signature submodule requires the users to revalidate and authenticates users, and presents a legal disclaimer to the users prior to the users signing into the databases so that the signature submodule provides an on-demand guarantee of fidelity of data in the databases.

13. The patient care management system of claim 10, wherein the signature submodule is configured to allow users to attach a digital signature in the databases associated with the test subjects, the case report forms, and the study visits.

14. The patient care management system of claim 10, wherein the query submodule is configured to allow data entry personnel to annotate data in the databases that fail any quality benchmark set in the multiple clinical trials.

15. The patient care management system of claim 1 wherein the disease is selected from the group consisting of dementia, depression, bipolar disorder, eating disorder, post-traumatic stress disorder, psychotic disorder, autism, generalized anxiety disorder, panic disorder, phobia, social anxiety disorder, mental health disorder, obsessive-compulsive disorder, delirium, delusional disorder, mood disorders, substance abuse, and medical-neurologic conditions.

16. The patient care management system of claim 1 wherein treatment of the disease is selected from the group consisting of prescribing medications to test subjects and providing in-house nursing care.

17. The patient care management system of claim 16, wherein the medications are selected from the group consisting of buspirone, trazodone, valproic acid, carbamazepine, gynecomastia, donepezil, galantamine, rivastigmine, memantine, aripiprazole, clozapine, haloperidol, olanzapine, quetiapine, risperidone, ziprasidone, Vyvanse, Ritalin, Adderall, and Dexedrine.

18. A patient care management system using a goal attainment scaling method for treatment of a disease in test subjects, the patient care management system comprising:
an Internet-based multi-tenanted electronic data collection platform for hosting and coordinating multiple clinical trials of test subjects in which the platform can be used independently or used in collaboration with third-party data collection tools, the platform comprising:
a customer identity access management module configured to authorize selective access to users onto the platform, wherein users include: system administrator users, clinical trial designer users, data entry personnel users, and data study reviewer users;
a system administrator module configured to host and to coordinate multiple clinical trials by providing a workspace and access to databases associated with the multiple clinical trials;
a study planning module configured to manage the databases,
wherein at least one of the databases includes initial details and clinical details, wherein the initial details include: a title field, a brief description field, and a language field, and wherein the clinical details include: an inclusion/exclusion criteria field, a study site field, a measurement unit field, a code list field, a forms field, a study event field, a study protocol field, and a user field;
a site management module configured to allow selectively authorized users to establish information about the multiple clinical trials and to link test subjects to the multiple clinical trials;
a form builder module configured to allow the clinical trial designer users to design case report forms for the multiple clinical trials, wherein the form builder module allows the clinical trial designer users to set workflow of the multiple clinical trials, to identify goals of the test subjects, to define attainment levels of the goals, to rank the goals, and to follow up on rankings of the goals, wherein the case report forms allow longitudinal data collection of goals and symptoms of the test subjects participating in the multiple clinical trials by capturing point in time data;
a study protocol module configured to allow the clinical trial designer users to define scheduled and unscheduled study visits for test subjects participating in the multiple clinical trials and to collect data associated with test subjects during the study visits;
a study user management module configured to allow the clinical trial designer users to add and assign data entry personnel users to read or write data in the databases and enable access to the database to the data study reviewer users; and
a data collection (data collection) module configured to provide a graphical user interface for use by the data entry personnel users and the data study reviewer users,
wherein the data collection module is configured to allow data entry personnel users to add test subjects and to collect data from the case report forms in which the data collection module is a primary point of interaction for the data entry personnel users to add test subjects and to collect or enter data in the databases of the multiple clinical trials and in the case report forms,
wherein the data collection module comprises a form display submodule, a signature submodule, and a query submodule in which the form display, signature, and query submodules are operatively coupled to each other,
wherein the form display submodule is configured to display data in GAS format so as to present an interactive GUI that guides users through the workflow of identified goals and to guide users through programmed prompts to assess progress of test subjects in achieving their respective identified goals in subsequent follow-up visits,
wherein the signature submodule is configured to allow users to attach a digital signature in the databases associated with the test subjects, the case report forms, and the study visits in which the signature submodule requires the users to revalidate and to authenticate users and presents a legal disclaimer to the users prior to the users signing into the databases so that the signature submodule provides an on-demand guarantee of fidelity of data in the databases,
wherein the query submodule is configured to allow data entry personnel users to annotate data in the databases that fails any quality benchmark set in the multiple clinical trials,
wherein the form display, signature, and query submodules of the data collection module are operatively coupled to each other, and
wherein the customer identity access management, system administrator, study planning, site management, form builder, study protocol, study user management, and data collection modules are operatively coupled to each other.

19. A goal attainment scaling method for diagnosis and treatment of a disease in test subjects, the goal attainment scaling method comprising:
hosting and coordinating multiple clinical trials of test subjects by using an Internet-based multi-tenanted electronic data collection platform that can be used independently or used in collaboration with third-party data collection tools, the platform comprising:
a customer identity access management module configured to authorize selective access to users onto the platform, wherein users include: system administrator users, clinical trial designer users, data entry personnel users, and data study reviewer users;
a system administrator module configured to host and to coordinate multiple clinical trials by providing a workspace and access to databases associated with the multiple clinical trials;
a study planning module configured to manage the databases,
wherein at least one of the databases includes initial details and clinical details, wherein the initial details include: a title field, a brief description field, and a language field, and wherein the clinical details include: an inclusion/exclusion criteria field, a study site field, a measurement unit field, a code list field, a forms field, a study event field, a study protocol field, and a user field;
a site management module configured to allow selectively authorized users to establish information about the multiple clinical trials and to link test subjects to the multiple clinical trials;
a form builder module configured to allow the clinical trial designer users to design case report forms for the multiple clinical trials, wherein the form builder module allows the clinical trial designer users to set workflow of the multiple clinical trials, to identify goals of the test subjects, to define attainment levels of the goals, to rank the goals, and to follow up on rankings of the goals, wherein the case report forms allow longitudinal data collection of goals and symptoms of the test subjects participating in the multiple clinical trials by capturing point in time data;
a study protocol module configured to allow the clinical trial designer users to define scheduled and unscheduled study visits for test subjects participating in the multiple clinical trials and to collect data associated with test subjects during the study visits;
a study user management module configured to allow the clinical trial designer to add and assign data entry personnel users to read or write data in the databases and to enable access to the database to the data study reviewer users; and
a data collection module configured to provide a graphical user interface for use by the data entry personnel users and the data study reviewer users,
wherein the data collection module is configured to allow data entry personnel users to add test subjects and to collect data from the case report forms in which the data collection module is a primary point of interaction for the data entry personnel users to add test subjects and to collect or enter data in the databases of the multiple clinical trials and in the case report forms,
wherein the data collection module comprises a form display submodule, a signature submodule, and a query submodule in which the form display, signature, and query submodules are operatively coupled to each other,
wherein the form display submodule is configured to display data in goal attainment scaling format so as to present an interactive GUI that guides users through the workflow of identified goals and to guide users through programmed prompts to assess progress of test subjects in achieving their respective identified goals in subsequent follow-up visits,
wherein the signature submodule is configured to allow users to attach a digital signature in the databases associated with the test subjects, the case report forms, and the study visits in which the signature submodule requires the users to revalidate and to authenticate users and presents a legal disclaimer to the users prior to the users signing into the databases so that the signature submodule provides an on-demand guarantee of fidelity of data in the databases,
wherein the query submodule is configured to allow data entry personnel users to annotate data in the databases that fails any quality benchmark set in the multiple clinical trials,
wherein the form display, signature, and query submodules of the data collection module are operatively coupled to each other, and
wherein the customer identity access management, system administrator, study planning, site management, form builder, study protocol, study user management, and data collection modules are operatively coupled together.

20. The goal attainment scaling method of claim 19, further comprising:
diagnosing the disease selected from the group consisting of dementia, depression, bipolar disorder, eating disorder, post-traumatic stress disorder, psychotic disorder, autism, generalized anxiety disorder, panic disorder, phobia, social anxiety disorder, mental health disorder, obsessive-compulsive disorder, delirium, delusional disorder, mood disorders, substance abuse, and medical-neurologic conditions; and
treating the disease with prescribed medications to test subjects in which the medications are selected from the group consisting of buspirone, trazodone, valproic acid, carbamazepine, gynecomastia, donepezil, Vyvanse, galantamine, rivastigmine, memantine, aripiprazole, clozapine, haloperidol, olanzapine, quetiapine, risperidone, ziprasidone, Ritalin, Adderall, and Dexedrine.
